(19) United States
(12) Patent Application Publication
(10) Pub. No.: US 2002/0055717 A1
(43) Pub. Date: May 9, 2002

(54) FLUID-BASED AGENT DELIVERY DEVICE WITH SELF-EXPANDING DELIVERY ELEMENT

(76) Inventor: PHILIPPE PONCET, FREMONT, CA (US)

Correspondence Address: SHELDON & MAK, 225 SOUTH LAKE AVENUE, 9TH FLOOR, PASADENA, CA, 91101

(21) Appl. No.: 08/968,756

(22) Filed: Oct. 20, 1997

(51) Int. Cl. A61M 5/178; A61M 5/00

(52) U.S. Cl. 604/170.03

(57) ABSTRACT

A device suitable for delivering a fluid-based agent in a body lumen in a mammalian body comprises a housing including an elongated bore, and a delivery element. The delivery element includes a proximal portion, a distal portion preferably comprised of a superelastic material, and a wall. The wall defines (i) a fluid passage extending between the proximal portion and the distal portion, and (ii) a plurality of apertures in fluid communication with the fluid passage at the distal portion. The housing is selectively movable relative to the delivery element between a first position in which the distal portion is disposed in the bore, and a second position in which the distal portion is disposed exteriorly of the bore. The distal portion is constrained inside the bore in the first position, and the distal portion self-expands in the second position. The apertures contact the vessel wall in the second position to enable the agent to be delivered directly to the wall of the lumen.

### BACKGROUND

The present invention is directed to a device and method for delivering a fluid-based agent to a selected site within the body and, more particularly, for delivering a fluid-based agent such as a pharmaceutical agent, diagnostic agent or preventative agent in a body lumen.

A challenging problem in the treatment of patients is the delivery of a fluid-based agent to only a selected local site within the body. For example, it is commonly desirable to achieve an effective concentration of a therapeutic or preventative agent at only a selected local site within a body lumen. The amount of an agent needed to effectively treat a disease in a particular organ can oftentimes only be achieved by establishing blood levels that can produce damaging side effects on other internal organs and healthy tissue. For example, therapy administered to prevent blood coagulation at one site can produce unwanted bleeding at other sites.

Devices and methods are known for delivering fluid-based agents locally into the body. For example, percutaneous transluminal coronary angioplasty balloon dilation catheters have been formed with drug coatings. These devices can be bulky and limit blood flow. There are also stents that include a polymer sheath with an incorporated controlled release drug. Such stents are less than fully satisfactory due to the size of the sheath and its limited compatibility with certain drugs.

There are known devices for delivering fluid-based agents in vessels that include an element having a portion that is preformed in a permanent coil shape as exemplified in U.S. Pat. No. 5,523,092 to Hanson et al. The disclosed device includes a delivery sheath having a diameter larger than the diameter of the coil, making the device intrusive inside the body. Other known devices such as disclosed in U.S. Pat. No. 5,603,694 to Brown et al. include an element that transforms from a coil shape to a more linear shape, for example, when heated by a heat source or manipulated by a guide wire inside the element while the element is inside the body. Such devices are also less than fully satisfactory in that the required heating of the element to remove the coil shape and enable removal of the element from the body, makes such devices complicated to operate inside the body. Also, depending on the transformation temperature of the element, the required heating cause the shape transformation can damage human tissue.

Thus, there is a need for a device and method for delivering a fluid-based agent in a lumen in a mammalian body that (i) can deliver the agent directly to substantially only the selected location, thereby reducing the amount of the agent that needs to be delivered such as to achieve a desired effect, (ii) reduces side effects on other internal organs and healthy tissue, (iii) is small sized and, thus, relatively less intrusive and less restrictive to blood flow than some known devices, and (iv) does not require an external heat source or guide wire to operate in the lumen.

### SUMMARY

The present invention is a device and method suitable for delivering a fluid-based agent into a lumen within a mammalian body that satisfies the above needs. The device is particularly suitable for delivering a fluid-based agent directly into a wall of a lumen. The lumen can be in a vessel or any other tissue that contains or transports fluid in the body. The fluid-based agent can be a therapeutic agent, a preventative agent or a diagnostic agent.

The device comprises a housing including an elongated bore, and a delivery element. The delivery element includes a proximal portion, a distal portion and a wall. The wall defines a fluid passage extending between the proximal portion and the distal portion. The wall also defines a plurality of apertures in fluid communication with the fluid passage at the distal portion.

The housing is selectively movable relative to the delivery element between a position in which the distal portion is disposed in the bore, and a position in which the distal portion is disposed extended from the bore. The bore is sized such that the distal portion assumes a constrained shape in the bore.

The distal portion self-expands to a different shape exteriorly of the bore. The distal portion self expands preferably to a helical shape. The diameter of the expanded distal portion increases such that the apertures are proximate to the wall of the lumen. Preferably, the apertures are in direct contact with the wall.

The present device can deliver a therapeutic agent, preventative agent or a diagnostic agent directly to the wall of the lumen, producing important advantages. Particularly, the device (i) reduces the amount of the agent needed to achieve a desired effect, (ii) reduces side effects on other tissue, and (iii) is small sized.

The distal portion of the delivery element is preferably formed of a superelastic material, which can be superelastically constrained when inside the bore of the housing and transform to the helical shape outside the bore. This transformation can be repeated without producing plastic deformation of the distal portion. The housing has a small diameter because the delivery element can be introduced into the body in a straightened shape and not in a preformed coil shape. The superelastic material is preferably capable of forming stress-induced martensite at temperatures near mammalian body temperature and recovering to the non-stressed shape in direct response to release of the applied stress. Accordingly, the present device does not require a heat source or a straightening element inside the element to transform the shape of the distal portion inside of the body, thus making the device simple to use.

### DRAWINGS

These and other features, aspects and advantages of the present invention will become better understood from the following description, appended claims and accompanying drawings, in which:

FIG. 1 illustrates a device according to the present device positioned in a body lumen with the delivery element in a constrained shape inside the bore of the housing;

FIG. 2 illustrates the device of FIG. 1 with the distal portion of the delivery element in a non-deformed shape exterior to the bore;

FIG. 3 is a perspective of the delivery element of FIG. 1 in the deformed shape; and

FIG. 4 is an enlarged cross-sectional view in the direction of line 4-4 of FIG. 3.

### DESCRIPTION

The present invention is a device and method for delivering a fluid-based agent in a lumen within a mammalian body. The lumen can be any natural tissue conduit that contains or transports body fluids. For example, the lumen can be in a vessel of the cardiovascular system such as a vein or artery, a bile duct, or a fluid conduit in the intestinal tract, urinary system or respiratory system. The device can be used in humans as well as in animals.

As used herein, the term “fluid-based agent” means any liquid or liquid-based agent. For example, fluid-based agents can include liquids, liquid suspensions, liquid emulsions, gels, suspensions, liquid mixtures and liquid/solid mixtures.

The present invention is particularly suitable for delivering therapeutic agents, preventative agents and diagnostic agents directly to body tissue. The tissue is typically the wall of a vessel or an organ at a selected delivery site. The selected site can be a diseased or healthy section of the fluid conduit.

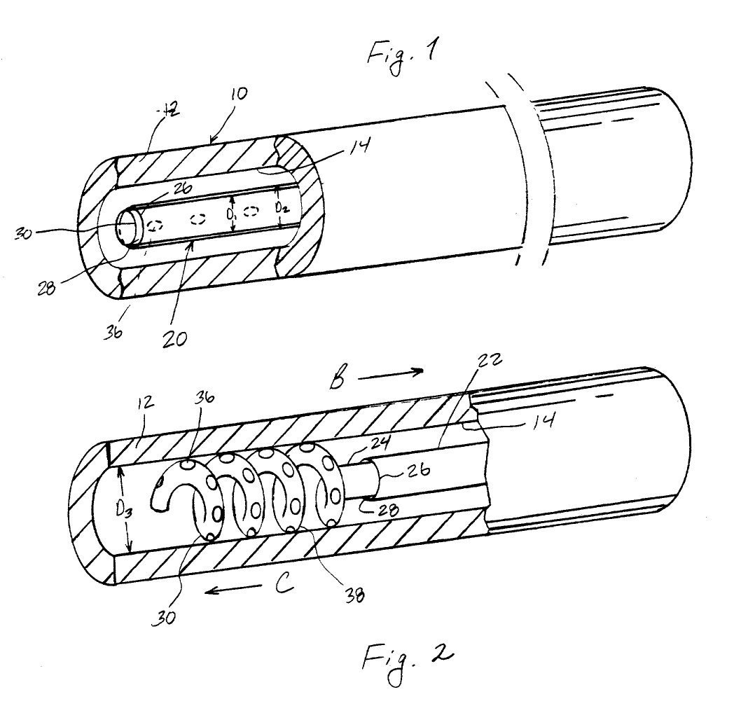

The device 20 according to the present invention is shown in FIG. 1 positioned in a body lumen 10 defined by a wall 12 having an inner surface 14. The device 20 comprises an elongated tubular housing 22 and a fluid-based agent delivery element 24. The housing 22 includes a proximal end (not shown), a distal end 26, and an elongated bore 28 which extends between the proximal end and the distal end 26. The delivery element 24 is shown inside the bore 28 in a constrained condition prior to the delivery of a fluid-based agent in the lumen 10. The delivery element 24 has a diameter D, in this condition which is typically from about 0.005 inches to about 0.01 inches.

The housing 22 has a outer diameter D₂ which is typically about 0.007 inches to about 0.02 inches, which is smaller than the diameter of the lumen 12 as defined by the inner diameter D₃ of the wall 12. For example, the lumen 12 can typically have an inner diameter of from about 0.08 inches to about 1 inch for vessels. Small vessels typically have a lumen diameter of from about 0.08 inches to about 0.25 inches. The housing 22 outer diameter can be varied depending on the size of the lumen in which the device 20 is used. The housing 22 has a sufficient length such that the proximal end extends outside of the body during use of the device 20 so that a user can manipulate the device 20. Accordingly, the housing 22 length can be varied depending on the distance between the point of entry of the device 20 into the body and the delivery site of the fluid-based agent.

The housing 22 can be formed of a suitable biocompatible material including metals such as stainless steel, and non-metallic materials such as polymers. The housing 22 has sufficient strength to constrain the delivery element 24 in the constrained condition in the bore 28 so that the delivery element 24 does not assume the recovered shape prior to being located at the treatment site. In addition, the housing 22 is capable of bending during advancement in body lumens to enable placement of the device 20 in tortuous fluid conduits.

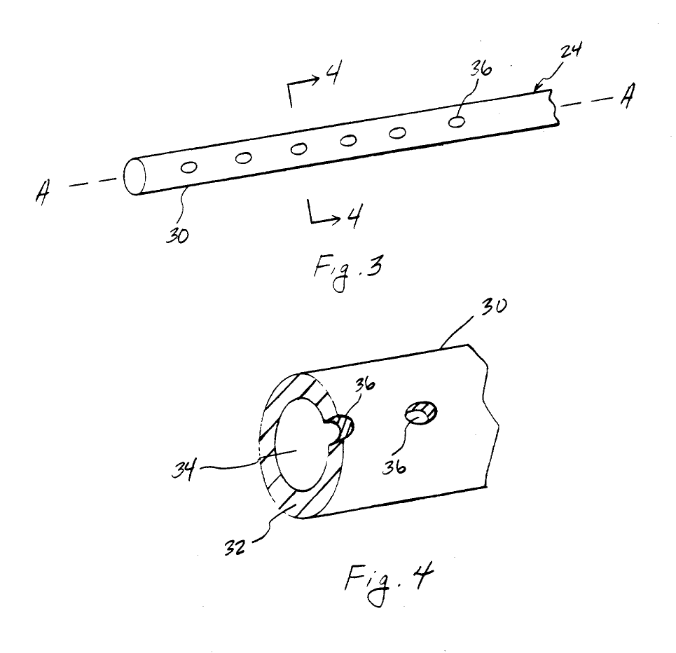

The delivery element 24 includes a proximal portion (not shown), a distal portion 30, and a wall 32 defining a fluid passage 34 (FIG. 4) extending between the proximal portion and the distal portion 30. A plurality of apertures 36 are formed through the wall 32 at the distal portion 30 in fluid communication with the fluid passage 34. As shown in FIG. 3, the apertures 36 can be substantially aligned with each other along a longitudinal axis A-A of the delivery element 24 in the deformed condition. The apertures 36 are typically formed along only one side of the wall 32 as shown. The apertures 36 typically have a diameter of about 0.001 inches to about 0.01 inches, and preferably have a diameter of from about 0.002 inches to about 0.004 inches. The aperture 36 size can be varied to control the rate of dispensing of the agent from the delivery element 24. The aperture 36 size can also be varied along the length of the distal portion 30 to achieve variable dispensing rates through the apertures 36 along the length of the distal portion 30. The apertures 36 can be circular or optionally have other shapes such as oval or rectangular. Conventional forming processes such as drilling and lasing can be used to form the apertures 36.

The distal portion 30 of the delivery element 24 is preferably comprised of a shape memory alloy that can be constrained in the constrained (straightened) shape inside the bore 28 of the housing 22, and then self-expand so as to assume a recovered shape when extended from the bore 28. The proximal portion of the delivery element 24 can be formed of a different material than the distal portion 30 as the proximal portion does not undergo the same shape transformation during use in the body lumen. The materials selected for the distal portion 30 and the proximal portion are preferably materials that are biocompatible and can remain in the body lumen during delivery of the fluid-based agent without damage to body organs and tissue, and also exhibit passive chemical behavior.

The shape memory material comprising the distal portion 30 of the delivery element 24 is preferably a superelastic material that can accomplish a shape change without having to undergo a temperature change as required for thermoelastic shape memory materials. Superelastic materials form stress-induced martensite when mechanically stressed at a temperature at least above Aₛ (austenite start), and preferably above A_{f} (austenite finish) The material is preferably a superelastic material having a large non-linear elastic range and capable of large strains without the occurrence of permanent deformation. Superelastic materials can be deformed substantially reversibly by 8% and more, by the application of mechanical stress and stress release. These properties enable the housing 22 to have a small bore 28 size.

Suitable superelastic materials for forming the distal portion 30 include, for example, binary Ni—Ti, and Ni—Ti alloys including elemental additions such as V, Fe, Nb, Co, Cr and Zr. Ni—Ti alloys are available that have an Mₛ temperature at near mammalian body temperature (about 35°-40° C.) and do not require heating by a heat source to cause a shape change when inside the body. The shape change between the constrained state and the recovered state is achieved by stress release. These alloys are also characterized as having a low modulus and high austenitic yield strength. Other suitable superelastic materials include copper-based alloys consisting essentially of Cu, Al and Zn; Cu, Al and Ni; and Cu and Zn.

The distal portion 30 is superelastically constrained in a first position of the housing 22 shown in FIG. 1. The diameter D₁ of the distal portion 30 approximately equals the inner diameter of the housing 22 so that the distal portion 30 assumes substantially the shape of the bore 28.

The housing 22 is movable relative to the delivery element 24 in the direction B (toward the proximal end) between the first position shown in FIG. 1 and a second position shown in FIG. 2. In the second position, the distal portion 30 of the delivery element 24 is disposed exteriorly of the bore 28 of the housing 22 and assumes a self-expanded shape. The self-expanded shape is more austenitic as the stress-induced martensite transforms to austenite in direct response to the release of the mechanical stress on the distal portion 30 exerted in the bore 28 by the housing 22. The recovered shape of the distal portion 30 is preferably helical as shown. The effective diameter of the distal portion 30 defined by the coil turns 38 is greater than the outer diameter of the housing 22. In the helical shape, at least some of the apertures 36 are on the side of the coil turns 38 that face the inner surface 14 of the wall 12. Preferably, each of the apertures 36 face the inner surface 14. This is achieved by forming the apertures 32 along only one side of the wall 12 as shown in FIG. 3. The diameter of the recovered distal portion 30 is preferably substantially equal to or slightly larger than the inner diameter D₃ of the wall 12 of the lumen 10, such that the apertures 36 directly contact the inner surface 14 of the wall 12 as shown in FIG. 2. This contact allows the agent to diffuse directly into the inner surface 14 at the locations of the apertures 36.

A fluid-based agent is introduced into the fluid passage 34 of the delivery element 24 at the proximal end of the device 20 and caused to flow to the distal portion 30. The agent can be introduced into the fluid passage 34 using a conventional fluid supply source by-pumping, injection or gravity flow. The delivery pressure of the agent into the fluid passage 34 can be varied to control the rate of dispensing through the apertures 36 in the distal portion 30.

The fluid-based agent can be a therapeutic agent, a diagnostic agent, a preventative agent or-another suitable agent as will be understood by those skilled in the art with reference to the disclosure herein. For example, suitable agents include anticoagulants such as heparin, agents which inhibit platelet formation, agents which effect platelet metabolic function, vascular cell growth promoters, vasodilators, cholesterol lowering substances, antibodies, dyes and markers.

A method of delivering a fluid-based agent in a body lumen in a mammalian body comprises introducing the device 20 into the body and into a selected lumen 10 with the housing 22 in the first position shown in FIG. 1. The device 20 is advanced in the lumen 10 until the distal portion 30 of the delivery element 24 is positioned slightly distally relative to a selected agent delivery site. It is preferred to position the distal portion 30 in this manner because the length of the distal portion 30 decreases as it transforms to the recovered shape. The housing 22 is then moved to the second position as shown in FIG. 2 such that the distal portion 30 of the delivery element is extended from the bore 28 of the housing 22 and superelastically self-expands to the second shape at the site. For more localized delivery of the fluid-based agent, less than the entire length of the distal portion 30 can be extended from the bore 28. The effective diameter D₃ of the coil turns is preferably slightly greater than the diameter of the lumen 10 so that the apertures 36 directly contact the wall 12. The superelastic material of the distal portion 30 exerts sufficient elastic force on the wall 12 such that the distal portion 30 supports the wall 12. The coil turns each have a small diameter approximately equal to D₃. The small diameter housing 22 and the distal portion 30 consequently present less disturbance to fluid flow through the lumen 10 than some known larger-sized devices. The selected fluid-based agent is introduced into the device 20 at the proximal end of the delivery element 24 and flowed via the fluid passage 34 from the proximal end to the distal portion 30. The agent is dispensed from the distal portion 30 via the apertures 36 into the lumen 10 at the site. The agent flows substantially directly into the wall 12 of the lumen 10 and not into the fluid stream in the lumen 10, where it can be carried to other locations in the body at which the agent is not required and can have potentially harmful side effects. The agent can typically be delivered in a predetermined volume within less than about 30 minutes after dispensing is initiated through the apertures 36.

After an effective amount of the agent is dispensed in the lumen, the distal portion 30 is retracted into the bore 28 of the housing 22 and transforms to the first shape shown in FIG. 1 by forming stress-induced martensite. The device 20 can then be relocated to other selected agent delivery sites in the same lumen, and the same or a different fluid-based agent can be dispensed at the other sites.

The effective amount of the agent is dependent on the type of agent delivered and the recommended dose of the agent. For example, a therapeutically effective amount of a therapeutic agent to treat a disease or condition can be delivered using the device 20. Preventative agents can be delivered in effective amounts to prevent the onset or progression of a disease or other undesirable state.

Thus, the present device can deliver fluid-based agents such as therapeutic and preventative agents directly to walls of fluid conduits such as vessels and organs. Diagnostic agents such as radioactive isotopes can also be delivered directly to the wall. In the preferred application, the device reduces the amount of the agent needed to achieve a desired effect because the agent substantially diffuses directly into the wall and does not enter into the fluid stream in the lumen. Consequently, the agent is delivered substantially to the selected site and is not carried to other internal organs and healthy tissue, thus reducing side effects of the agent.

In addition, the device 20 is small sized due to the use of the superelastic delivery element 30 and the small sized housing 22. Consequently, the device 20 is less restrictive to the flow of blood and other fluids as compared to larger known devices. The small size of the device 20 also makes it relatively easy to maneuver in lumens. Furthermore, the device 20 is relatively simple to use because it does not require the simultaneous manipulation of a guide wire and/or a heating device to cause the delivery element 24 to change shape.

The present invention has been described in considerable detail with reference to certain preferred embodiments thereof, however, other embodiments are possible. Therefore, the spirit and scope of the appended claims should not be limited to the description of the preferred embodiments contained herein.

What is claimed is:

1. A device for delivering a fluid-based agent in a lumen, in a mammalian body, the device comprising:
a) a housing including an elongated bore having a diameter, the housing being sized to fit in a lumen in a mammalian body; and
b) a delivery element including a proximal portion, a distal portion, and a wall defining (i) a fluid passage extending between the proximal portion and the distal portion and (ii) a plurality of apertures in fluid communication with the fluid passage at the distal portion, the housing being selectively movable relative to the delivery element between a first position in which the distal portion is disposed in the bore and a second position in which the distal portion extends exteriorly of the bore;
wherein the distal portion is constrained in a first shape in the first position, and the distal portion self-expands to a second shape having a diameter greater than the diameter of the bore in response to the housing being moved to the second position.

2. The device of claim 1, wherein the distal portion of the delivery element has a diameter approximately equal to the diameter of the bore in the first shape.

3. The device of claim 1, wherein the lumen (i) is defined by a wall and (ii) has a diameter, the second shape of the distal portion has a diameter at least substantially equal to the diameter of the lumen such that at least some of the apertures contact the wall in the second position.

4. The device of claim 1, wherein the second shape of the distal portion is helical.

5. The device of claim 1, wherein the delivery element has a longitudinal axis along which the apertures are longitudinally spaced from each other in the first shape of the delivery element.

6. The device of claim 1, wherein the distal portion of the delivery element is comprised of a superelastic material.

7. The device of claim 1, wherein the distal portion of the delivery element is comprised of a superelastic material which superelastically forms stress-induced martensite at about the mammalian body temperature in the first shape.

8. A device for delivering a fluid-based agent in a lumen in a mammalian body, the device comprising:
a) a housing including an elongated bore having a diameter, the housing being sized to fit in a lumen in a mammalian body; and
b) a delivery element including a proximal portion, a distal portion and a wall defining (i) a fluid passage extending between the proximal portion and the distal portion and (ii) a plurality of apertures in fluid communication with the fluid passage at the distal portion, the housing being movable relative to the delivery element between a first position in which the distal portion is disposed in the bore and a second position in which the distal portion extends exteriorly of the bore;
wherein (i) the distal portion of the delivery element is comprised of a superelastic material which superelastically forms stress-induced martensite at about the mammalian body temperature, and (ii) the distal portion assumes a superelastically constrained first shape in the first position, and the distal portion superelastically transforms to a second shape having a diameter greater than the diameter of the bore in response to the housing being moved to the second position.

9. The device of claim 8, wherein the distal portion of the delivery element has a diameter approximately equal to the diameter of the bore in the first shape.

10. The device of claim 8, wherein the lumen (i) is defined by a wall and (ii) has a diameter, the second shape of the distal portion has a diameter at least substantially equal to the diameter of the lumen such that at least some of the apertures contact the wall in the second position.

11. The device of claim 8, wherein the second shape of the distal portion is helical.

12. The device of claim 8, wherein the delivery element has a longitudinal axis along which the apertures are longitudinally spaced from each other in the first shape.

13. The device of claim 8, wherein the distal portion of the delivery element is comprised of a Ni—Ti alloy.

14. A device for delivering a fluid-based agent to a wall of a lumen in a mammalian body, the device comprising:
a) a housing defining an elongated bore having a diameter, the housing being sized to fit in a lumen in a mammalian body; and
b) a delivery element including a proximal portion, a distal portion comprised of a superelastic material that superelastically forms stress-induced martensite at about mammalian body temperature, and a wall defining (i) a fluid passage extending between the proximal portion and the distal portion and (ii) a plurality of apertures in fluid communication with the fluid passage at the distal portion, the housing being movable relative to the delivery element between a first position in which the distal portion is disposed in the bore and a second position in which the distal portion extends exteriorly of the bore;
wherein (i) the distal portion assumes a superelastically constrained shape having a diameter approximately equal to the diameter of the bore in the first position, and (ii) the distal portion superelastically transforms to a second shape in response to the housing being moved to the second position, the second shape having a diameter greater than the diameter of bore such that at least some of the apertures contact the wall of the lumen, thereby enabling the fluid-based agent to be delivered directly to the wall of the lumen via the apertures.

15. The device of claim 14, wherein the delivery element has a longitudinal axis along which the apertures are longitudinally spaced from each other in the first position.

16. The device of claim 14, wherein the distal portion of the delivery element is comprised of a Ni—Ti alloy.

17. A method of delivering a fluid-based agent in a lumen in a mammalian body, the method comprising the steps of:
a) introducing the device of claim 1 into a lumen in a mammalian body with the housing in the first position;
b) advancing the device in the lumen until the distal portion of the delivery element is positioned approximately at a site;
c) moving the housing such that at least a portion of the distal portion of the delivery element is extended from the elongated bore and transforms to the second shape at the site; and
d) delivering a fluid-based agent through the fluid passage and the apertures of the delivery element and into the lumen at the site.

18. The method of claim 17, wherein the second shape of the distal portion of the delivery element is substantially helical.

19. The method of claim 17, wherein the distal portion is comprised of a superelastic material.

20. The method of claim 17, wherein the distal portion of the delivery element is comprised of a Ni—Ti containing alloy which superelastically forms stress-induced martensite at about the mammalian body temperature.

21. The method of claim 17, wherein the distal portion of the delivery element is sized in the second shape such that at least some of the apertures contact a wall defining the lumen, such that the fluid-based agent is delivered directly to the wall via the apertures.

22. The method of claim 17, where the fluid-based agent is selected from the group consisting of therapeutic agents, preventative agents and diagnostic agents.

23. The method of claim 21, wherein the fluid-based agent is a therapeutic agent and the step of delivering comprises delivering a therapeutically effective amount of the therapeutic agent to the wall at the site.

24. The method of claim 21, wherein the fluid-based agent is a preventative agent and the step of delivering comprises delivering a preventative effective amount of the preventative agent to the wall at the site.

25. The method of claim 17, further comprising the steps of:
moving the housing to the first position such that the distal portion assumes the first shape; and
advancing the device in the lumen until the distal portion is positioned at another site.

26. The method of claim 17, wherein the step of moving comprises extending substantially the entire distal portion from the elongated bore.

27. A method of delivering a fluid-based agent to a wall of a lumen in a mammalian body, the method comprising the steps of:
a) introducing the device of claim 14 into a lumen in a mammalian body with the housing in the first position;
b) advancing the device in the lumen until at least a portion of the distal portion of the delivery element is positioned at a site;
c) moving the housing to the second position such that the distal portion of the delivery element superelastically transforms to the second shape at the site; and
d) delivering a pharmaceutical agent through the fluid passage and the apertures of the delivery element and to the wall of the lumen at the site.

28. The method of claim 27, wherein the second shape of the distal portion of the delivery element is helical.

29. The method of claim 27, wherein the distal portion of the delivery element is comprised of a Ni—Ti alloy that superelastically forms reversible stress-induced martensite at about the mammalian body temperature.

30. The method of claim 27, further comprising the steps of:
moving the housing to the first position such that the distal portion of the delivery element superelastically transforms to the first shape; and
advancing the device in the lumen until the distal portion is positioned at another site.

31. The method of claim 27, wherein the step of moving comprises extending substantially the entire distal portion from the elongated bore.
